(19) United States
(12) Patent Application Publication
(10) Pub. No.: US 2007/0185393 A1
(43) Pub. Date: Aug. 9, 2007

(54) SYSTEM FOR MEASURING VITAL SIGNS USING AN OPTICAL MODULE FEATURING A GREEN LIGHT SOURCE

(75) Inventors: Zhou Zhou, La Jolla, CA (US); Michael James Thompson, San Diego, CA (US); Matthew John Banet, Del Mar, CA (US)

Correspondence Address: Triage Wireless, Inc.;Matthew John Banet, 6540 LUSK BLVD., C200, SAN DIEGO, CA, 92121, US

(73) Assignee: TRIAGE WIRELESS, INC., San Diego, CA (US)

(21) Appl. No.: 11/307,375

(22) Filed: Feb. 3, 2006

(51) Int. Cl. A61B 5/00; A61B 5/02

(52) U.S. Cl. 600/323; 600/485; 600/500

(57) ABSTRACT

The invention provides a system for measuring vital signs from a patient that includes: 1) a first sensor including a first electrode that measures a first electrical signal from the patient; 2) a second sensor including a second electrode that measures a second electrical signal from the patient; and 3) a third sensor including an optical system with a light source configured to emit green radiation and a photodetector configured to measure the green radiation emitted from the light source, after it irradiates the patient, to generate an optical signal; and 4) a controller that receives and processes the first and second optical and electrical signals and the electrical waveform to determine the patient's vital signs.

### BACKGROUND OF THE INVENTION

The present invention relates to a system for measuring vital signs, particularly blood pressure, featuring an optical system.

### Description of Related Art

Pulse oximeters are medical devices featuring an optical module, typically worn on a patient's finger or ear lobe, and a processing module that analyzes data generated by the optical module. The optical module typically includes first and second light sources (e.g., light-emitting diodes, or LEDs) that transmit optical radiation at, respectively, red (λ˜630-670 nm) and infrared (λ˜800-1200 nm) wavelengths. The optical module also features a photodetector that detects the transmitted radiation reflected from an underlying artery. Typically the red and infrared LEDs sequentially emit radiation that is partially absorbed by blood flowing in the artery. The photodetector is synchronized with the LEDs to detect the transmitted radiation. In response, the photodetector generates a separate radiation-induced signal corresponding to each wavelength. The signal, called a plethysmograph, varies in a time-dependent manner as each heartbeat varies the volume of arterial blood and hence the amount of radiation absorbed along the path of light between the LEDs and the photodetector. A microprocessor in the pulse oximeter digitizes and processes plethysmographs generated by the red and infrared radiation to determine the degree of oxygen saturation in the patient's blood using algorithms known in the art. A number between 94%-100% is considered normal, while a value below 85% typically indicates the patient requires hospitalization. In addition, the microprocessor analyzes time-dependent features in the plethysmograph to determine the patient's heart rate.

Various methods have been disclosed for using pulse oximeters to obtain arterial blood pressure. One such method is disclosed in U.S. Pat. No. 5,140,990 to Jones et al., for a ‘Method Of Measuring Blood Pressure With a Photoplethysmograph’. The '990 Patent discloses using a pulse oximeter with a calibrated auxiliary blood pressure measurement to generate a constant that is specific to a patient's blood pressure.

Another method for using a pulse oximeter to measure blood pressure is disclosed in U.S. Pat. No. 6,616,613 to Goodman for a ‘Physiological Signal Monitoring System’. The '613 Patent discloses processing a pulse oximetry signal in combination with information from a calibrating device to determine a patient's blood pressure.

Asmar, U.S. Pat. No. 6,511,436, and Golub, U.S. Pat. Nos. 5,857,795 and 865,755, each disclose a method and device for measuring blood pressure that processes a time difference between points on an optical plethysmograph and an electrocardiogram along with a calibration signal.

Chen et al, U.S. Pat. No. 6,599,251, discloses a system and method for monitoring blood pressure by detecting pulse signals at two different locations on a subject's body, preferably on the subject's finger and earlobe. The pulse signals are preferably detected using pulse oximetry devices, and then processed to determine blood pressure.

### BRIEF SUMMARY OF THE INVENTION

In one aspect, the invention provides a system for measuring vital signs (e.g. blood pressure) from a patient that features: i) a first sensor including a first electrode that measures a first electrical signal from the patient; ii) a second sensor including a second electrode that measures a second electrical signal from the patient; and iii) a third sensor including an optical system with a light source configured to emit green radiation between 510 and 590 nm and a photodetector configured to measure the green radiation emitted from the light source, after it irradiates the patient, to generate an optical signal. To process the electrical and optical signals, the system additionally includes a controller (e.g., a microcontroller or microprocessor) that runs a computer algorithm configured to: i) receive and process the first and second electrical signals to generate an electrical waveform; ii) receive and process the optical signal to generate an optical waveform; and iii) calculate a time difference between a first feature on the electrical waveform and a second feature on the optical waveform to determine a blood pressure for the patient.

In preferred embodiments, the light source is an LED or diode laser configured to emit green radiation between 510 and 590 nm. Optical systems which use light sources in this spectral region are referred to herein as ‘green optical systems’. In other preferred embodiments, the optical system is configured to operate in a reflection-mode geometry, e.g. both the light source and photodetector are disposed on a same side of the substrate (e.g., a printed circuit board). In this case the photodetector is aligned to detect radiation first emitted from the light source and then reflected from the patient's tissue to generate the optical waveform.

In other embodiments the optical system is included in a patch configured to be worn on the patient's body. The patch may include an adhesive component configured to adhere to the patient's skin. In this case, the first and second electrodes may also be included in separate patches or the same patch, and the optical system may also include a third electrode.

Alternatively, in other embodiments, the optical system and electrodes are housed within a hand-held or body-worn unit. In this configuration these sensors are typically oriented to measure electrical and optical signals from at least one of the patient's fingers. In still other embodiments, the controller additionally includes an amplifier system (e.g. a two-stage amplifier system) configured to process the first and second electrical signals to generate an electrical waveform. The controller can also use this same amplifier system, or a different amplifier system, to process the optical signals to generate an optical waveform.

In an alternate embodiment, calibration parameters are based on biometric data, e.g., height, arm span, weight, body mass index, age. The calibration parameters may are not specific to an individual patient, but rather determined for a general class of patients. For example, the calibration parameters are based on correlations between blood pressure and features in the optical or electrical waveforms observed in the analysis of clinical data sets. Conjunctively, the calibration parameters may be based on correlations between biometric parameters and features in the optical or electrical waveforms observed in the analysis of clinical data sets.

In embodiments, the microprocessor or microcontroller within the controller runs computer code or ‘firmware’ that determines blood pressure by processing: 1) a first time-dependent feature of the optical waveform; 2) a second time-dependent feature of the electrical waveform; and 3) a calibration parameter. In this case the calibration parameter is determined by a conventional device for measuring blood pressure, such as a blood pressure cuff.

In other embodiments, the system features a first light source that emits green radiation to generate a first optical waveform, and a second light source that emits infrared radiation to generate a second optical waveform. In this case the controller runs computer code or firmware that processes the first and second optical waveforms to generate a pulse oximetry value using techniques that are known in the art. In a related embodiment, the controller can run computer code or firmware that processes the optical waveform to generate a heart rate value. In yet another embodiment, the controller can run computer code or firmware that processes the first and second electrical signals to generate an ECG waveform, which can then be processed to calculate a heart rate.

The invention has many advantages. In particular, through use of an optical system operating in a reflection-mode geometry and based on a green light source, the invention measures optical waveforms that are relatively insensitive to motion-related artifacts and have a high signal-to-noise ratio, particularly when compared to waveforms measured using red or infrared radiation in a similar geometry. Ultimately this means waveforms measured with the invention, when processed in concert with an electrical waveform to determine a time difference, result in an accurate blood pressure measurement that can be made from nearly any part of a patient's body. Measurements can be made with a disposable patch sensor or hand-held device.

In a more general sense, the invention provides a single, low-profile, disposable system that measures a variety of vital signs, including blood pressure, without using a cuff. This and other information can be easily transferred to a central monitor through a wired or wireless connection to better characterize a patient. For example, with the system a medical professional can continuously monitor a patient's blood pressure and other vital signs during their day-to-day activities. Monitoring patients in this manner minimizes erroneous measurements due to ‘white coat syndrome’ since the monitor automatically and continuously makes measurements away from a medical office with basically no discomfort to the patient. Using the system of the invention, information describing the patient's blood pressure can be viewed using an Internet-based website, personal computer, or a mobile device. Blood-pressure information measured continuously throughout the day provides a relatively comprehensive data set compared to that measured during isolated medical appointments. For example, this approach identifies trends in a patient's blood pressure, such as a gradual increase or decrease, which may indicate a medical condition that requires treatment. Measurements can be made completely unobtrusive to the patient. The monitor is easily worn by the patient during periods of exercise or day-to-day activities, and makes a non-invasive blood-pressure measurement in a matter of seconds. The resulting information has many uses for patients, medical professional, insurance companies, pharmaceutical agencies conducting clinical trials, and organizations for home-health monitoring.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF THE DRAWINGS

FIG. 1A is a schematic top view of an adhesive patch sensor that combines an electrical system with a green optical system to measure blood pressure and other vital signs according to the invention;

FIG. 1B is a schematic, cross-sectional view of the patch sensor of FIG. 1A;

FIG. 2A is a schematic view of the patch sensor system of FIG. 1A in electrical contact with a belt-worn controller;

FIG. 2B is a schematic view of the patch sensor system of FIG. 2A attached to a patient;

FIG. 3 is a graph of time-dependent optical and electrical waveforms generated by the patch sensor system of FIG. 1A;

FIG. 4 is a graph of various time-dependent optical waveforms measured using the green optical system of FIG. 1A;

FIG. 5 is a schematic diagram of a two-stage amplifier system used to amplify signals generated by the green optical system of FIG. 1A; and

FIG. 6 is a graph of time-dependent optical waveforms amplified by the first and second stages of the two-stage amplifier of FIG. 5.

### DETAILED DESCRIPTION OF THE INVENTION

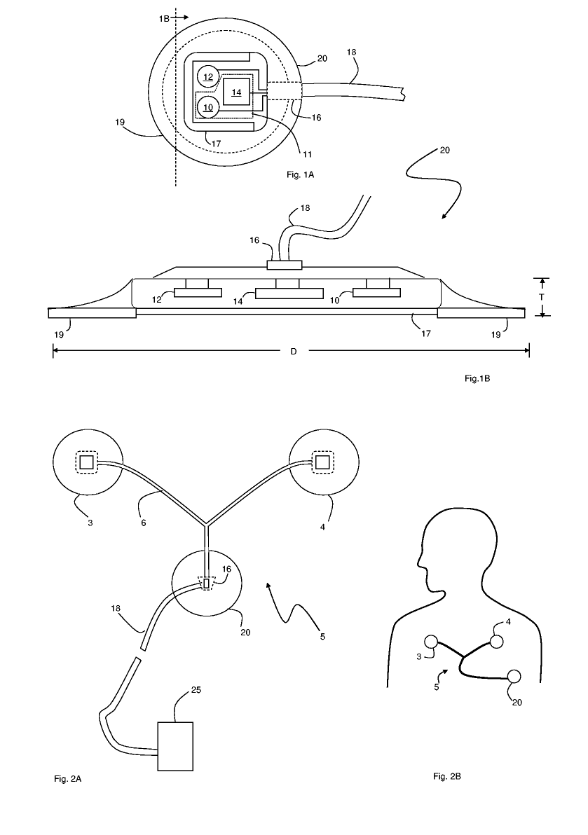

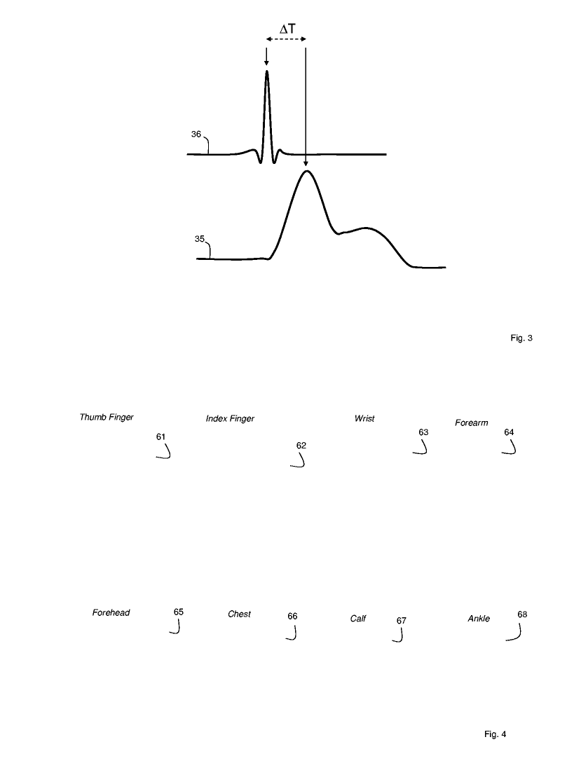

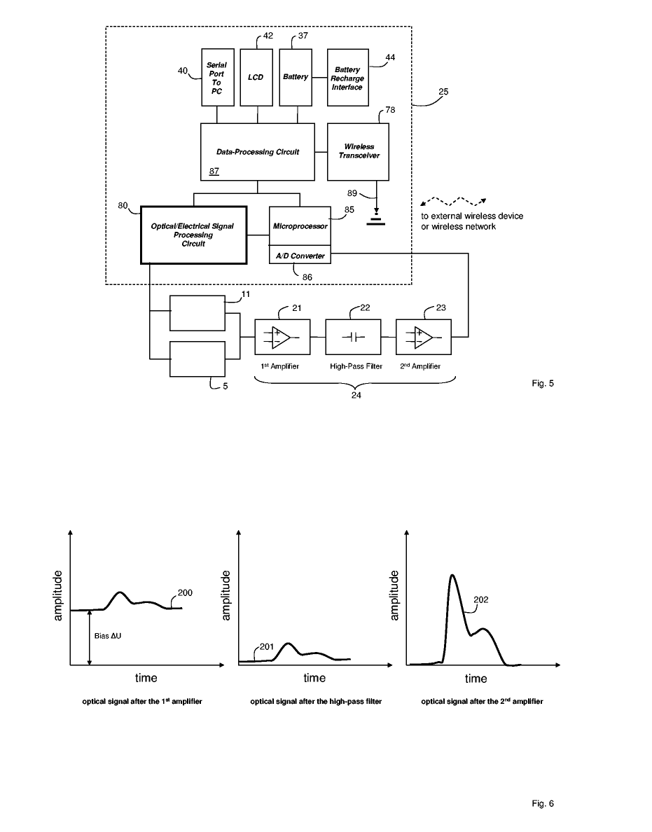

FIGS. 1A, 1B, 2A and 2B show an adhesive patch sensor 20 that makes a cuffless measurement of blood pressure according to the invention by measuring an optical waveform (35 in FIG. 3) and an electrical waveform (36 in FIG. 3). A data-processing module 25 connected to the patch sensor 20 then calculates a time difference ΔT between specific portions these two waveforms (e.g., their peaks) to determine blood pressure. To improve the accuracy of the blood pressure measurement, the patch sensor 20 features a green LED 10 (typically emitting a wavelength between 510-590 nm, and more typically emitting a wavelength between 550-590 nm) and photodetector 14 that combine to form a ‘green optical system’ 11 operating in a reflection-mode geometry. Because of the optical absorption and reflective properties of a patient's skin and underlying arteries, the reflective green optical system 11 measures a strong, stable optical waveform from nearly any portion of the patient's body. FIG. 4, for example, illustrates this point by showing optical waveforms 61-68 collected with a reflective green optical system from body portions ranging from the forehead (optical waveform 65) to the ankle (optical waveform 68). Moreover, optical waveforms collected in a reflection-mode geometry with the green optical system, when compared to waveforms measured using red or infrared LEDs in a similar geometry, are relatively insensitive to motion-related artifacts and have a high signal-to-noise ratio. Ultimately this means that these waveforms, when processed in concert with an electrical waveform to determine ΔT, result in an accurate blood pressure measurement that can be made from nearly any part of a patient's body with a comfortable, adhesive patch sensor.

Measurements of optical waveforms using a green optical system are described in more detail in Weijia Cui et al., ‘In Vivo Reflectance of Blood and Tissue as a Function of Light Wavelength’, IEEE Transactions on Biomedical Engineering, 37(6), 632-639, (1990), the contents of which are incorporated herein by reference.

The patch sensor 20 can additionally include an infrared LED 12 that radiates infrared radiation which can also be detected by the photodetector 14 to generate a separate optical waveform. Using techniques known in the art, the data-processing module 25 can independently analyze AC and DC components of optical waveforms generated by the green 10 and infrared 12 LEDs to determine a patient's blood oxygen saturation. To measure the electrical waveform, the patch sensor 20 includes a metal, horseshoe-shaped electrode 17 that surrounds the green 10 and infrared 12 LEDs and the photodetector 14. The horseshoe-shaped electrode 17 measures an electrical signal, and connects through a Y-shaped cable 6 to second 3 and third 4 electrodes that measure separate electrical signals. These electrical signals pass through the Y-shaped cable 6, a second cable 18, and ultimately to a two-stage amplifier circuit within the data-processing module 25. There, the electrical signals are amplified and filtered to generate the electrical waveform. The second cable 18 also ports optical signals generated by the green 10 and infrared 12 LEDs to the two-stage amplifier circuit, where they too are amplified and filtered to generate a processed optical waveform. An algorithm running on this module, described in more detail below, can calculate a patient's systolic and diastolic blood pressure, heart rate, and pulse oximetry by analyzing the processed optical and electrical waveforms. The patch sensor 20 also features an adhesive component 19 that adheres to the patient's skin to secure the LEDs 10, 12, photodetector 14, and electrode 17. This allows the patch sensor to operate in a reflection-mode geometry, and additionally minimizes the effects of motion which may reduce the accuracy of the blood pressure measurement.

During operation, the second cable 18 snaps into a plastic header 16 disposed on a top portion of the patch sensor 20. Both the cable 18 and header 16 include matched electrical leads that supply power and ground to the LEDs 10, 12, photodetector 14, and additionally supply an electrical connection between the electrodes 17, 3, 4 and the two-stage amplifier circuit within the data-processing module 25. When the patch sensor 20 is not measuring optical and electrical waveforms, the cable 18 unsnaps from the header 16, while the sensor 20 remains adhered to the patient's skin. In this way a single sensor can be used for several days. After use, the patient removes and then discards the sensor 20. The patch sensor 20 preferably has a diameter, ‘D’, ranging from 0.5 centimeter (‘cm’) to 10 cm, more preferably from 1.5 cm to 3.0 cm, and most preferably 2.5 cm. The patch sensor 20 preferably has a thickness, ‘T’, ranging from 1.0 millimeter (“mm”) to 3 mm, more preferably from 1.0 mm to 1.5 mm, and most preferably 1.25 mm, and preferably includes a body composed of a polymeric material such as a neoprene rubber. The body is preferably colored to match a patient's skin color, and is preferably opaque to reduce the affects of ambient light. The body is preferably circular in shape, but can also be non-circular, e.g. an oval, square, rectangular, triangular or other shape.

Referring to FIG. 2B, the patch sensor 20 and second 3 and third 4 electrodes form a patch sensor system 5 that is typically worn on a patient's chest. Typically the second 3 and third 4 electrodes are adhered on each side of the patient's heart, and the patch sensor 20 is adhered to the patient's shoulder or arm. In a preferred embodiment, the patch sensor 20 is adhered as close to the patient's hand as possible, as this increases the ΔT separating peaks in the optical and electrical waveforms, thereby increasing the resolution of the blood pressure measurement. For the purposes of measuring blood pressure as described herein, the electrodes within the patch sensor system only need to collect electrical signals required to generate an electrical waveform found in a conventional ECG obtained from two electrodes. These electrodes can therefore be placed on the patient at positions that differ from those used during a standard multi-lead ECG (e.g., positions used in ‘Einthoven's Triangle’).

FIG. 3 shows both the optical 35 and electrical 36 waveforms generated by, respectively, the electrodes and green optical system in the patch sensor system. Following a heartbeat, electrical impulses travel essentially instantaneously from the patient's heart to the electrodes, which detect it to generate the electrical waveform 36. At a later time, a pressure wave induced by the same heartbeat propagates through the patient's arteries, which are elastic and increase in volume due to the pressure wave. Ultimately the pressure wave arrives at a portion of the artery underneath the optical system, where light-emitting diodes and a photodetector detect it by measuring a time-dependent change in optical absorption to generate the optical waveform 35. The propagation time of the electrical impulse is independent of blood pressure, whereas the propagation time of the pressure wave depends strongly on pressure, as well as mechanical properties of the patient's arteries (e.g., arterial size, stiffness). The microprocessor runs an algorithm that analyzes the time difference ΔT between the arrivals of these signals, i.e. the relative occurrence of the optical 35 and electrical 36 waveforms as measured by the patch sensor. Calibrating the measurement (e.g., with a conventional blood pressure cuff) accounts for patient-to-patient variations in arterial properties, and correlates ΔT to both systolic and diastolic blood pressure. This results in a calibration table. During an actual measurement, the calibration source is removed, and the microprocessor analyzes ΔT along with other properties of the optical and electrical waveforms and the calibration table to calculate the patient's real-time blood pressure.

To better determine ΔT, both the optical and electrical waveforms can be ‘fit’ using a mathematical function that accurately describes the waveform's features, and an algorithm (e.g., the Marquardt-Levenberg algorithm) that iteratively varies the parameters of the function until it best matches the time-dependent features of the waveform. Moreover, using this technique, blood pressure-dependent properties of the waveform, such as its width, rise time, fall time, and area, can be calibrated as described above. After the calibration source is removed, the patch sensor measures these properties along with ΔT to determine the patient's blood pressure. Alternatively, the waveforms can be filtered using mathematical techniques, e.g. to remove high or low frequency components that do not correlate to blood pressure. In this case the waveforms can be filtered using well-known Fourier Transform techniques to remove unwanted frequency components.

Methods for processing the optical and electrical waveform to determine blood pressure are described in the following co-pending patent applications, the entire contents of which are incorporated by reference: 1) CUFFLESS BLOOD-PRESSURE MONITOR AND ACCOMPANYING WIRELESS, INTERNET-BASED SYSTEM (U.S. Ser. No. 10/709,015; filed Apr. 7, 2004); 2) CUFFLESS SYSTEM FOR MEASURING BLOOD PRESSURE (U.S. Ser. No. 10/709,014; filed Apr. 7, 2004); 3) CUFFLESS BLOOD PRESSURE MONITOR AND ACCOMPANYING WEB SERVICES INTERFACE (U.S. Ser. No. 10/810,237; filed Mar. 26, 2004); 4) VITAL-SIGN MONITOR FOR ATHLETIC APPLICATIONS (U.S. Ser. No. ______ filed Sep. 13, 2004); 5) CUFFLESS BLOOD PRESSURE MONITOR AND ACCOMPANYING WIRELESS MOBILE DEVICE (U.S. Ser. No. 10/967,511; filed Oct. 18, 2004); 6) BLOOD PRESSURE MONITORING DEVICE FEATURING A CALIBRATION-BASED ANALYSIS (U.S. Ser. No. 10/967,610; filed Oct. 18, 2004); 7) PERSONAL COMPUTER-BASED VITAL SIGN MONITOR (U.S. Ser. No. 10/906,342; filed Feb. 15, 2005); 8) PATCH SENSOR FOR MEASURING BLOOD PRESSURE WITHOUT A CUFF (U.S. Ser. No. 10/906,315; filed Feb. 14, 2005); 9) SMALL-SCALE, VITAL-SIGNS MONITORING DEVICE, SYSTEM AND METHOD (U.S. Ser. No. 10/907,440; filed Mar. 31, 2005); 10) PATCH SENSOR SYSTEM FOR MEASURING VITAL SIGNS (U.S. Ser. No. 11/160957; filed Jul. 18, 2005); 11) WIRELESS, INTERNET-BASED SYSTEM FOR MEASURING VITAL SIGNS FROM A PLURALITY OF PATIENTS IN A HOSPITAL OR MEDICAL CLINIC (U.S. Ser. No. 11/162719; filed Sep. 20, 2005); 12) HAND-HELD MONITOR FOR MEASURING VITAL SIGNS (U.S. Ser. No. 11/162742; filed Sep. 21, 2005); and 13) CHEST STRAPP FOR MEASURING VITAL SIGNS (U.S. Ser. No. 11/306243; filed Dec. 20, 2005).

FIG. 4 shows sample optical waveforms 61-68 measured from various areas on a patient's body using the green optical system described above. While the waveforms vary in intensity, each clearly shows pulses corresponding to individual heart beats. This indicates that the green optical system, when combined with the above-described system for measuring electrical waveforms, can make effective measurements of blood pressure from virtually any part of the patient's body. Optical waveforms measured from the thumb 61 and index finger 62 yield the strongest signals, while those measured from the calf 67 and ankle 68 yield weaker signals. Measurements from the wrist 63, forearm 64, forehead 65 and chest 66 yield signals between these two extremes.

FIG. 5 shows a preferred configuration of electronic components featured within the data-processing module 25. A data-processing circuit 87 connects to an optical/electrical signal processing circuit 80 that controls the LED and photodetector within the green optical system 11, as well as the three electrodes within the patch sensor system 5. During operation, signals from both the green optical system 11 and the electrodes within patch sensor system 5 independently pass through a two-stage amplifier system 24 that includes first 21 and second 23 amplifier stages separated by a high-pass filter 22. The first 21 and second 23 amplifiers independently amplify optical signals generated by the green optical system 11 along with electrical signals generated by electrodes within the patch sensor system 5. The high-pass 22 filter removes low-frequency noise, as well as DC component in the signal, from these signals to further improve signal quality. Signals that pass through the two-stage amplifier system 24 are then sent to the analog-to-digital converter 86 embedded within the microprocessor. The analog-to-digital converter 86 digitizes both the optical and electrical waveforms to generate arrays of data points that can be processed by the microprocessor using the algorithms described above to determine blood pressure, heart rate, and pulse oximetry.

To communicate with external wireless devices and networks, the data-processing circuit 87 connects to a wireless transceiver 78 that communicates through an antenna 89 to a matched transceiver embedded within an external component. The wireless transceiver 78 can be a short-range wireless transceiver, e.g. a device based on 802.11, Bluetooth™, Zigbee™, or part-15 wireless protocols. Alternatively, the wireless transceiver 78 can be a cellular modem operating on a nation-wide wireless network, e.g. a GSM or CDMA wireless network. The data-processing circuit 87 can also display information on a liquid crystal display (‘LCD’) 42, and transmit and receive information through a serial port 40. A battery 37 powers all the electrical components within the processing module, and is preferably a metal hydride battery (generating 3-7 V, and most preferably about 3.7 V) that can be recharged through a battery-recharge interface 44.

FIG. 6 illustrates the benefits of the two-stage amplifier system shown in FIG. 5. The first amplifier stage amplifies both the DC and AC components of the optical waveform detected by the photodetector to generate a first amplified waveform 200. The first amplified waveform 200 includes an AC signal portion representing a time-dependent heart beat, along with a DC bias (ΔU) resulting from, e.g., reflected, scattered and ambient radiation detected by the photodetector. The signal 200 is sent to the analog-to-digital converter 86 embedded within the microprocessor 85 and is processed by the microprocessor using the algorithms described above to determine blood pressure, heart rate, and pulse oximetry. The first amplified signal 200 passes through the high-pass filter to remove the DC bias while preserving the AC signal portion, resulting in a second amplified signal 201. This signal 201 then passes through the second amplifier stage to further amplify the AC signal portion to generate the third amplified signal 202. This final amplifier stage further increases the amplitude of the waveform, thereby improving the accuracy of the blood pressure measurement.

In an alternate embodiment of the invention, the data-processing module and patch sensor are used within a hospital, and the data-processing module includes a short-range wireless link (e.g., a module operating Bluetooth™, 802.11a, 802.11b, 802.1g, or 802.15.4 wireless protocols) that sends vital-sign information to an in-hospital wireless network. In this case the in-hospital wireless network may connect to a computer system that processes signals from the patch sensor to determine its location. For example, in this embodiment, a nurse working at a central nursing station can quickly view the vital signs and location of the patient using a simple computer interface.

In still other embodiments, electronics associated with the data-processing module (e.g., the microprocessor) are disposed directly on the patch sensor, e.g. on the circuit board that supports the optical system. In this configuration, the circuit board may also include a display to render the patient's vital signs. In another embodiment, a short-range radio (e.g., a Bluetooth™, 802.15.4, or part-15 radio) is mounted on the circuit board and wirelessly sends information (e.g., optical and electrical waveforms; calculated vital signs such as blood pressure, heart rate, pulse oximetry, ECG, and associated waveforms) to an external controller with a matched radio, or to a conventional cellular telephone or wireless personal digital assistant. Or the short-range radio may send information to a central computer system (e.g., a computer at a nursing station), or though an internal wireless network (e.g. an 802.11—based in-hospital network). In yet another embodiment, the circuit board can support a computer memory that stores multiple readings, each corresponding to a unique time/date stamp. In this case, the readings can be accessed using a wireless or wired system described above.

In still other embodiments, blood pressure may be determined in a way that does not require the determination of an electrical waveform 36 and pulse transit time (ΔT in FIG. 3) by using one or more optical systems with one or more light sources configured to emit green radiation. In such an embodiment, blood pressure is determined using features in the optical waveforms alone (e.g., pulse waveform width, rise time, fall time, distribution, area). Alternatively, differences in the aforementioned features from two or more optical waveforms observed at different positions on the patient's body could be used to determine blood pressure.

In still other embodiments, the patch sensor can include sensors in addition to those described above, e.g. sensors that measure temperature, motion (e.g. an accelerometer), or other properties. Or the sensor system can interface with other sensors, such as a conventional weight scale.

In still other embodiments, information measured by the patch sensor is sent through a wired or wireless connection to an Internet-based website.

Still other embodiments are within the scope of the following claims.

What is claimed is:

1. A system for measuring vital signs from a patient, comprising: a first sensor comprising a first electrode that measures a first electrical signal from the patient; a second sensor comprising a second electrode that measures a second electrical signal from the patient; a third sensor comprising an optical system comprising a light source configured to emit green radiation between 510-590 nm and a photodetector configured to measure green radiation emitted from the light source after it irradiates the patient to generate an optical signal; and a controller comprising a system configured to: i) receive and process the first and second electrical signals to generate an electrical waveform; ii) receive and process the optical signal to generate an optical waveform; and iii) calculate a time difference between a first feature on the electrical waveform and a second feature on the optical waveform to determine a blood pressure for the patient.

2. The system of claim 1, wherein the light source is an LED.

3. The system of claim 1, wherein the light source is configured to emit green radiation between 510 and 590 nm.

4. The system of claim 1, wherein the third sensor is configured to operate in a reflection-mode geometry.

5. The system of claim 1, wherein the third sensor further comprises a substrate, and the light source and photodetector are disposed on a same side of the substrate.

6. The system of claim 5, wherein the photodetector is aligned to detect radiation first emitted from the light source and then reflected from the patient's tissue to generate the optical waveform.

7. The system of claim 1, wherein the third sensor is comprised by a patch configured to be worn on the patient's body.

8. The system of claim 7, wherein the patch further comprises an adhesive component configured to adhere to the patient's body.

9. The system of claim 1, wherein the third sensor further comprises a third electrode.

10. The system of claim 9, wherein the first sensor is a first adhesive patch comprising the first electrode, and the second sensor in a second adhesive patch comprising the second electrode.

11. The system of claim 1, wherein the first, second, and third sensors are comprised by a hand-held unit.

12. The system of claim 11, wherein the hand-held unit further comprises first and second sensors configured to measure electrical signals from at least one of the patient's fingers.

13. The system of claim 11, wherein the hand-held unit further comprises a third sensor configured to measure an optical signal from at least one of the patient's fingers.

14. The system of claim 1, wherein the controller further comprises a first amplifier system configured to process the first and second electrical signals to generate an electrical waveform.

15. The system of claim 1, wherein the controller further comprises a second amplifier system configured to process the optical signals to generate an optical waveform.

16. The system of claim 1, wherein the controller further comprises an algorithm that determines blood pressure by processing: 1) a first time-dependent feature of the optical waveform; 2) a second time-dependent feature of the electrical waveform; and 3) a set of calibration parameters.

17. The system of claim 1, wherein the third sensor further comprises a first light source that emits green radiation that generates a first optical waveform, and a second light source that emits infrared radiation that generates a second optical waveform.

18. The system of claim 17, wherein the controller further comprises an algorithm that processes the first and second optical waveforms to generate a pulse oximetry value.

19. The system of claim 1, wherein the controller further comprises an algorithm that processes the optical waveform to generate a heart rate value.

20. The system of claim 1, wherein the controller further comprises an algorithm that processes the first and second electrical signals to generate an ECG waveform.

21. The system of claim 20, wherein the controller further processes the ECG waveform to calculate a heart rate.

22. A system for measuring vital signs from a patient, comprising: a first electrode comprised by a first adhesive patch and configured to measure a first electrical signal from the patient; a second electrode comprised by a second adhesive patch and configured to measure a second electrical signal from the patient; a third sensor comprised by an adhesive patch and comprising an optical system comprising a light source configured to emit green radiation between 510-590 nm and a photodetector configured to measure green radiation reflected off the patient to generate an optical signal; and a controller comprising a system configured to: i) receive and process the first and second electrical signals to generate an electrical waveform; ii) receive and process the optical signal to generate an optical waveform; and iii) calculate a time difference between a first feature on the electrical waveform and a second feature on the optical waveform to determine a blood pressure for the patient.

23. A hand-held system for measuring vital signs from a patient, comprising: a housing comprising: a first electrode configured to measure a first electrical signal from the patient; a second electrode configured to measure a second electrical signal from the patient; a third sensor comprising an optical system comprising a light source configured to emit green radiation between 510-590 nm and a photodetector configured to measure green radiation reflected off the patient to generate an optical signal; and a controller comprising a system configured to: i) receive and process the first and second electrical signals to generate an electrical waveform; ii) receive and process the optical signal to generate an optical waveform; and iii) calculate a time difference between a first feature on the electrical waveform and a second feature on the optical waveform to determine a blood pressure for the patient.
